(19) United States
(12) Patent Application Publication
(10) Pub. No.: US 2012/0053453 A1
(43) Pub. Date: Mar. 1, 2012

(54) MEDICAL NAVIGATION SYSTEM

(76) Inventor: Rainer Graumann, Hochstadt (DE)

(21) Appl. No.: 13/319,292

(22) PCT Filed: May 7, 2010
(86) PCT No.: PCT/EP2010/056269

§ 371 Date: Nov. 7, 2011

(30) Foreign Application Priority Data
May 13, 2009 (DE) 10 2009 021 025.3

(51) Int. Cl. A61B 5/05

(52) U.S. Cl. 600/424

(57) ABSTRACT

A medical navigation system for electromagnetic position and/or location determination of a sensor coil, which is located in a navigation space, includes a number of navigation modules, each having at least one field coil and a position coil. In each module, the sensor coil and the position coil have a fixed and known spatial relationship with each other. Each navigation module spans a navigation volume, with the respective navigation volumes in combination forming the navigation space. The individual navigation modules are arranged such that the position coil of a first of the navigation modules is located within the navigation volume of at least one other of the navigation modules. The first of the navigation modules and the other of the navigation modules are connected by a communication link.

### BACKGROUND OF THE INVENTION

1. Field of the Invention

The invention concerns a medical navigation system for electromagnetic position and attitude determination of a field coil or, respectively, sensor coil present in a navigation space.

2. Description of the Prior Art

Numerous electromagnetic navigation systems are used in modern medical workstations. These normally include at least one field generator to generate an electromagnetic field with a known field distribution, a sensor coil for position and attitude determination of a subject, and a common control and evaluation unit with which the field detected by the sensor coil is converted into position and attitude coordinates. The sensor coil normally includes a triplet of coils arranged at right angles to one another that detect the respective field components in one of the three spatial directions. For example, such a sensor coil can be integrated into a manually guided medical instrument so that the user can monitor and track the movements implemented with this instrument (on a monitor, for example). Navigation systems are typically “standalone” apparatuses, i.e. modules composed of field generator and field coil to which the sensor coil is connected with a cable. Such navigation systems can be flexibly integrated into a medical workstation.

The navigation volume spanned by such a navigation module—thus that volume in which attitude and position of a sensor coil can be determined—is typically a cube with an edge length of only approximately 30 to 50 cm. For example, this navigation volume is thus relatively small in comparison to a patient bed. Before a medical measure—for example a diagnosis, biopsy or operation that should take place with navigation assistance—the navigation module is therefore positioned in proximity to the region affected by the measure. Alternatively, multiple modules can be used simultaneously to enlarge the navigation volume, such that a larger navigation volume is created as a whole.

In order to enable an error-free position and/or attitude determination of the sensor coil within the navigation space composed of the navigation volumes of the individual modules, it is necessary that the position of the individual modules relative to one another is known. For this reason either the individual modules are attached at known positions, or their relative positions among one another are measured with suitable assistive means.

However, such a navigation system composed of multiple individual navigation modules is either less flexible, or a complicated and laborious determination of the relative position of the individual modules is necessary.

### SUMMARY OF THE INVENTION

An object of the present invention is to provide a medical navigation system for electromagnetic position and/or attitude determination that is flexible with regard to the combination of multiple navigation modules.

The medical navigation system according to the invention for electromagnetic position and/or attitude determination of a sensor coil present in a navigation space has multiple navigation modules that each include at least one field coil and a position coil. The field coil and the position coil of each navigation module have a fixed and known spatial relationship to one another. Each field coil of each navigation module spans a navigation volume; the navigation volumes of the individual navigation modules together form the navigation space. The individual navigation modules are now arranged such that the position coil of a first of the navigation modules lies within the navigation volume of at least one other navigation module among the multiple. Moreover, at least the first navigation module and the other navigation module are connected with one another via a communication link.

The conception of the medical navigation system according to the invention is based on the following considerations:

Only a relatively small navigation volume can be spanned with a single navigation module. The available navigation space can be enlarged via the combination of multiple modules. The sum of the navigation volumes generated by the individual navigation modules is designated as a navigation space, wherein overlapping regions of the individual navigation volumes are only taken into account once rather than twice. In other words, the navigation space is the envelope of the navigation volumes generated by the individual navigation modules. However, individual navigation modules can only be combined with one another when their position relative to one another is known. This necessity hinders the flexible composition of a medical workstation since the individual navigation modules must either be placed at fixed, known positions, or their positions relative to one another must be determined in a complicated procedure.

According to the invention, each of the navigation modules is equipped with a position coil that corresponds to a sensor coil in terms of its function, with the spatial arrangement between this position coil and the field coil of the respective navigation module being fixed and known. Moreover, because the individual navigation modules are arranged relative to one another such that the position coil of a first navigation module lies within the navigation volume of at least one other navigation module, and the individual navigation modules are networked among one another, it is possible to determine their relative position. For this purpose the individual navigation modules are connected to a central control and evaluation unit. This unit is configured to determine the position of the position coil of the first module in the navigation volume of the additional navigation module. Since the spatial relationship between the position coil of this first module and its field coil is known, the coordinate systems of the individual navigation modules can be calibrated. An attitude and position determination of the sensor coil in the entire navigation space spanned by the navigation modules is thus possible.

The navigation modules of the medical navigation system can be combined with one another as needed since their position relative to one another can be determined and therefore is known; the navigation space can be nearly arbitrarily increased by adding additional navigation modules.

It is particularly advantageous that, in accordance with the invention, a positioning of the navigation modules at fixed, known locations is superfluous; the complicated determination of the positions relative to one another is likewise not needed. The navigation system according to the invention enables the flexible assembly of a medical workstation; for example, an operating table into which a navigation module is integrated can be combined with an x-ray C-arm apparatus which likewise has such a navigation module.

According to a first embodiment, the individual navigation modules as well as the sensor coil are connected by a communication link (which can be wired or wireless) with a central control and evaluation unit. According to a further embodiment, the individual navigation modules are associated with different medical apparatuses. Such a medical navigation system allows a flexible assembly of a medical workstation. Because the necessary medical apparatuses were arranged according to practical medical considerations, the navigation system can be placed in operation immediately without incurring additional complicated preparation tasks.

According to a further embodiment, different carrier frequencies are provided for the field coils of the individual navigation modules. Given attitude and position determination of the sensor coil within the navigation space, it is necessary that which navigation module is to be associated with the signal received by the sensor coil can be established. This is possible in a simple manner by the use of different carrier frequencies. The attitude and position of the sensor coil that are initially detected in the coordinate system of the corresponding navigation module can thus be associated with the common coordinate system of the navigation space.

In the aforementioned navigation systems, the attitude of a moved sensor coil is determined in a static electromagnetic field. Conversely, in the medical navigation system that is now addressed the attitude and position of a movable field coil is now detected by static sensors.

An alternative medical navigation system for electromagnetic position and attitude determination has the following features:

Such a medical navigation system comprises a plurality of receiver modules as navigation modules which respectively comprise at least one sensor coil. The field coil—whose position is determined—spans a navigation space, wherein the receiver modules are arranged such that at least two receiver modules are arranged in the navigation space spanned by the field coil. The field coil and the receiver modules are connected with one another by a communication link.

An enlargement of the navigation volume can advantageously be achieved with the aforementioned medical navigation system, just as with the medical navigation systems described further above. By suitable arrangement of the receiver modules—for example along a line—the field coil can be handed off from receiver module to receiver module, such that a larger and nearly arbitrarily expandable navigation space is created in which the position of the field can be determined.

### BRIEF DESCRIPTION OF THE DRAWINGS

FIG. 1 illustrates a medical navigation system in accordance with the invention, in a plan view.

FIG. 2 illustrates the medical workstation of FIG. 1 in a perspective view.

FIG. 3 schematically illustrates a further embodiment of a medical navigation system in accordance with the invention, in a plan view.

### DESCRIPTION OF THE PREFERRED EMBODIMENTS

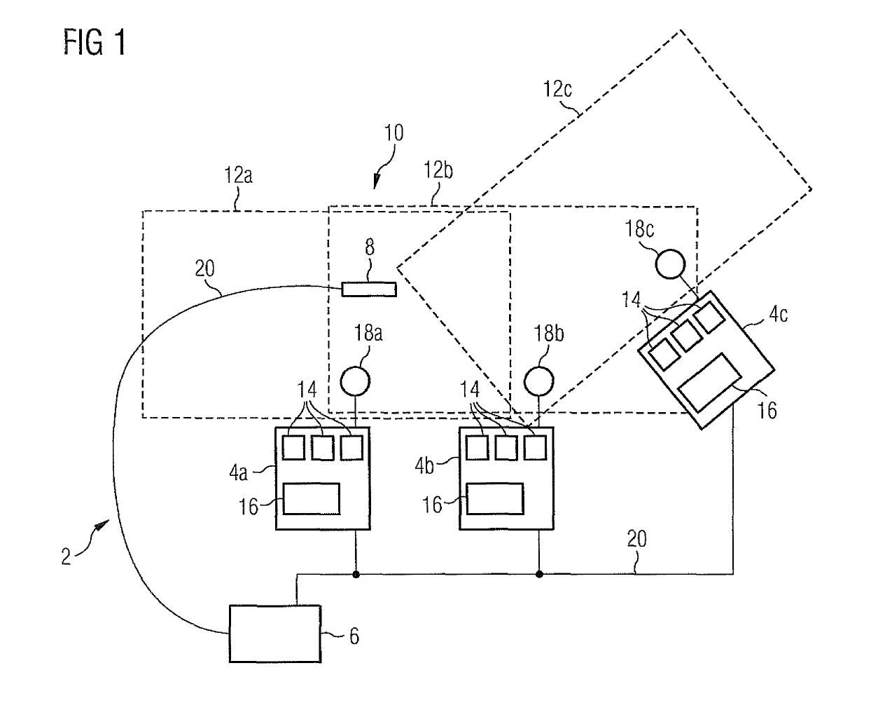

The medical navigation system 2 shown in FIG. 1 has three navigation modules 4a, 4b and 4c, a control and evaluation unit 6 connected with these and a sensor coil 8 whose attitude and position is determined in a navigation space 10 spanned by the three navigation modules 4a, 4b and 4c. The navigation modules 4a, 4b and 4c respectively span navigation volumes 12a, 12b and 12c. The navigation space 10 results as an envelope of the sum of the individual navigation volumes 12a, 12b and 12c. In FIG. 1 this envelope corresponds to a line running along the outer edges of the navigation volumes 12a, 12b and 12c. Typical dimensions of the navigation volumes 12a, 12b and 12c. spanned by the individual navigation modules 4a, 4b and 4c lie in a range from 30 to 50 cm. To generate the respective navigation volumes 12a, 12b and 12c, the navigation modules 4a, 4b and 4c respectively have the three field coils 14 that generate an electromagnetic field in the respective navigation volume 12a, 12b and 12c. The field coils 14 are operated by a field generator 16 likewise integrated into the navigation module 4a, 4b and 4c.

Each of the navigation modules 4a, 4b and 4c comprises a position coil 18a, 18b and 18c with which the position of the associated navigation module 4a, 4b and 4c relative to the other navigation modules 4a, 4b and 4c can be determined. In the following this is explained as an example for the navigation module 4b:

The position coil 18b of the navigation module 4b lies in the navigation volume 12c spanned by the navigation module 4c. With the use of the control and evaluation unit 6, the electromagnetic field generated by the navigation module 4c in the navigation volume 12c is determined at the location of the position coil 12b and the attitude of this position coil 12b is calculated in the navigation volume 12c, thus relative to the position of the navigation module 4c. The communication between the navigation modules 4a, 4b and 4c and the central control and evaluation unit 6 takes place via a wired or wireless communication link 20. Since the spatial arrangement of the position coil 18b relative to the navigation module 4b is known, the position of the navigation module 4b can be calculated starting from the position of the navigation module 4c. The position of the navigation module 4a (whose position coil 18a lies in the navigation volume 12b of the navigation module 4b) can analogously be determined starting from the position of the navigation module 4b. The positions of all navigation modules 4a, 4b and 4c relative to one another can be determined in the described manner.

Starting from the now-known positions of the navigation modules 4a, 4b and 4c relative to one another, a common coordinate system in the entire navigation space 10 can be established, The position of the sensor coil 9 can be represented in this common navigation system, regardless of where this resides in the navigation space 10. The necessary conversion—for example of the position coordinates of the sensor coil 8 that are measured in the navigation volume 12a with the aid of the navigation module 4a into the coordinates of the common coordinate system—takes place via the control and evaluation unit 6.

However, such a conversion requires that it can be established in which navigation volume 12a, 12b or 12c the sensor coil 8 is presently located. Two preferred operating methods of the medical navigation system 2 essentially suggest themselves here. A first possibility is to activate the field coils 14 of the individual navigation modules 4a, 4b and 4c sequentially. Since the activation of the field coils of the individual navigation modules 4a, 4b and 4c takes place via the control and evaluation unit 6, this is in the position to associate the respective values measured by the sensor coil 8 with one of the navigation modules 4a, 4b and 4c (and thus with one of the navigation volumes 12a, 12b or 12c) without any problems. According to a further preferred operating method, the field coils 14 of the navigation modules 4a, 4b and 4c are operated with different carrier frequencies so that the control and evaluation unit 6, using this information, is in the position to establish in which of the navigation volumes 12a, 12b or 12c the sensor coil 8 is presently located.

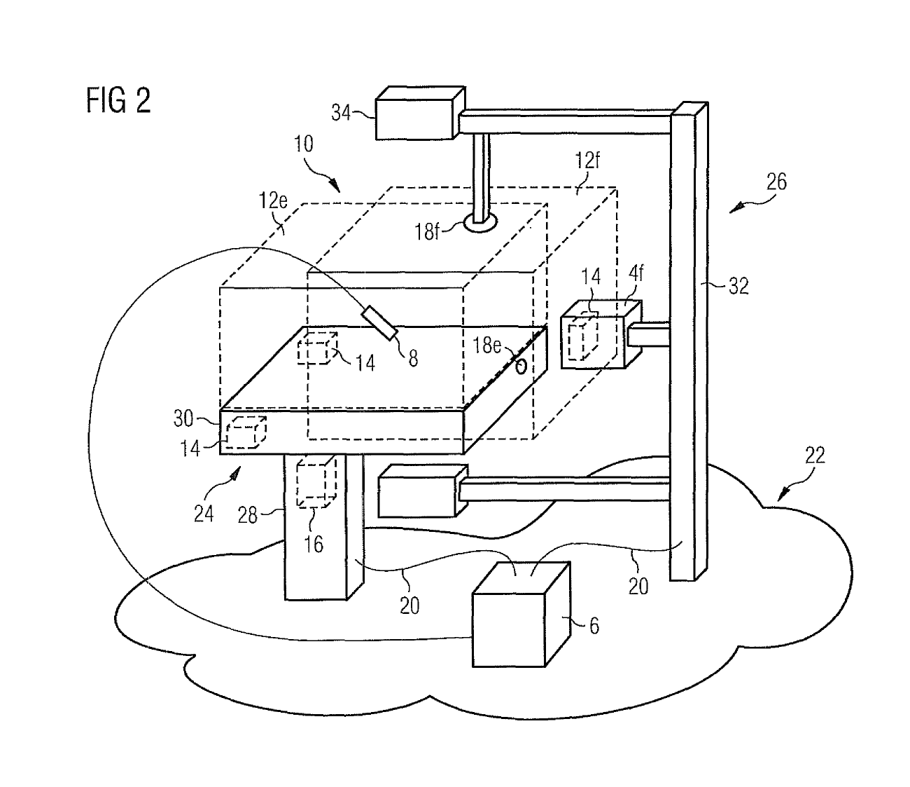

FIG. 2 shows a medical workstation 22 in a schematic perspective view. According to a further exemplary embodiment, a patient table 24 and an x-ray C-arm apparatus 26 are combined with one another. The patient table 24 has an integrated navigation module; a field generator 16 is thus located in the standing column 28; two field coils 14 are integrated into the table plate 30 of the patient table 24. With the aid of the field coils 14, a navigation volume 12e is generated which extends above the table plate 30. A position coil 18e is likewise integrated in a fixed manner into the table plate 30 of the patient table 24.

In addition to the typical elements of an x-ray C-arm apparatus 26 that are attached to a standing column (such as x-ray tube 34 and detector 36), in the x-ray apparatus 26 shown in FIG. 2 a navigation module 4f is additionally attached to its standing column 32. The field coil 14 of this navigation module 4f spans a navigation volume 12f which at least partially overlaps the navigation volume 12e spanned by the patient table 24. Moreover, at the x-ray C-arm apparatus 26 a position coil 18f is located in a fixed spatial position relative to the navigation module 4f.

The patient table 24 and the x-ray C-arm apparatus 26 can be flexibly assembled into the medical workstation 22 shown in FIG. 2. In order to enable a navigation in the complete navigation space 10 that is formed from the navigation volumes 12e, 12f of the patient table 24 and the x-ray C-arm apparatus 26, the two mechanical apparatuses are connected with a central control and evaluation unit 6 via a communication link 20.

The calibration of the coordinate systems of the navigation module integrated into the patient table 24 with that of the x-ray C-arm apparatus 26 takes place via position determination of the position coil 18e of the patient table 24 in the navigation volume 12f which is spanned by the navigation module 4f of the x-ray C-arm apparatus 26. Naturally, the position of the position coil 18f of the x-ray C-arm apparatus 26 can also conversely be determined in the navigation volume 12e spanned by the navigation module of the patient table 24. After a calibration of the coordinate systems between patient table 24 and x-ray C-arm apparatus 24 has taken place, the position of the sensor coil 8 can be determined in the entire navigation space 10.

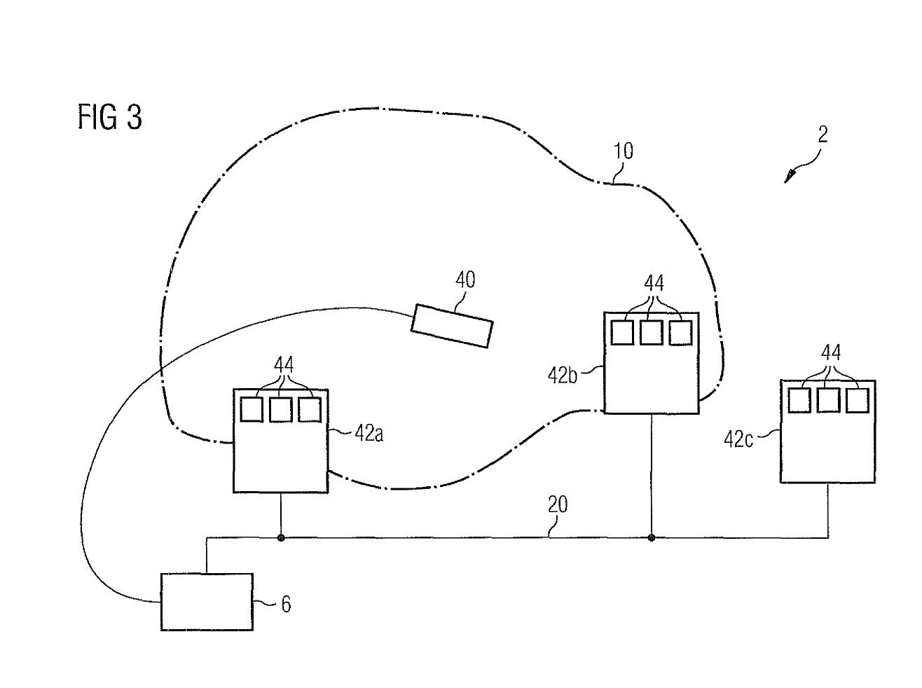

FIG. 3 shows a schematic plan view of a medical navigation system 2 according to an additional exemplary embodiment. In contrast to the aforementioned exemplary embodiments, the position of a field coil 40 is determined in the navigation system 2 shown here, and not the position of a sensor coil in a navigation space 10. The medical navigation system 2 moreover comprises three receiver coils 42a, 42b and 42c which are structurally identical to the navigation modules 4a through 4f shown in FIG. 1, except for the absent field generators 16. The coils that have previously been used as field coils now serve as receiver coils 44. The receiver coils 44a, 44b and 44c are connected with a central control and evaluation unit 6 via a communication link 20, advantageously with a cable. At least two receiver modules 42a, 42b and 42c are present in the navigation space 10 generated by the field coil 40. The receiver modules 42a, 42b and 42c can be flexibly arranged just like the navigation modules 4a through 4f, wherein only the position of one of the receiver modules 42a, 42b and 42c must be known as a reference position.

For example, if the position of the receiver module 42a is known, the control and evaluation unit 6 can determine the position of the field coil 40 using the field strength measured across the receiver coils 44 of the receiver module 42a, which field strength is generated by the field coil 40 in the navigation space 10. Since the receiver module 42b is likewise situated within the navigation space 10, the position of the field coil 40 relative to the receiver module 42b can also be determined at the same point in time. Since the position of the first receiver module 42a is known and the position of the field coil 40 can be determined relative to both this receiver module 42a and the receiver module 42b, the position of the receiver module 42b can be concluded from these values of the position of the receiver module 42a.

For example, if—due to the movement of a medical instrument with which the field coil 40 is connected—this is moved to the right in the exemplary embodiment shown in FIG. 3, the receiver module 42c arrives in the navigation space 10 spanned by the field coil 40. The position of the receiver module 42c relative to the receiver module 42b can now be determined just as was described for the receiver modules 42a and 42b.

Although modifications and changes may be suggested by those skilled in the art, it is the intention of the inventor to embody within the patent warranted heron all changes and modifications as reasonably and properly come within the scope of his contribution to the art.

I claim as my invention:

1-5. (canceled)

6. A medical navigation system comprising:
a sensor coil;
a plurality of navigation modules, each of said navigation modules comprising at least one field coil, and a position coil, the position coil in each of the navigation modules having a fixed and known spatial relationship to the at least one field coil in that navigation module;
each navigation module spanning a navigation volume and the respective navigation volumes of the plurality of navigation modules together forming a navigation space that is configured to encompass at least a portion of a region in which a medical procedure is implemented;
said plurality of navigation modules being arranged to cause the position coil of a first of the navigation modules to be within the navigation volume of at least one other of the navigation modules;
said first of said navigation modules and said at least one other of said navigation modules being connected with each other by a communication link;
said sensor coil detecting an electromagnetic field in said navigation space generated by said plurality of navigation modules; and
a computerized control and evaluation unit in communication with said sensor coil that identifies a position and an orientation of said sensor coil from the detected electromagnetic field.

7. A medical navigation system as claimed in claim 6 wherein said plurality of navigation modules are in communication via a further communication link with said control and evaluation unit.

8. A medical navigation system as claimed in claim 6 wherein individual navigation modules, in said plurality of navigation modules, are respectively associated with different medical apparatuses.

9. A medical navigation system as claimed in claim 6 wherein the respective field coils of the individual navigation modules in said plurality of navigation modules operate at respectively different carrier frequencies.

10. A medical navigation system comprising:
a field coil that emits an electromagnetic field that at least partially encompasses a region in which a medical procedure is implemented;
a plurality of receiver modules each configured to operate as a navigation module, each of said receiver modules comprising at least one sensor coil;
said field coil spanning a navigation space and said plurality of receiver modules being arranged to cause at least two of said receiver modules to be in the navigation space spanned by the field coil;
said field coil and said receiver modules being connected with each other by a communication link; and
a computerized control and evaluation unit in communication with at least said receiver modules that determines a position and orientation of said field coil from sensor signals emitted by the respective sensor coils due to interaction of the sensor coils with said electromagnetic field generated by said field coil.
